(19) United States
(12) Patent Application Publication
(10) Pub. No.: US 2026/0301170 A1
(43) Pub. Date: Oct. 1, 2026

(54) COMPUTATIONAL ANALYSIS OF RECTAL TUMOR PATHOLOGY FEATURES TO DETERMINE RESPONSE TO CHEMORADIATION

(71) Applicants: Case Western Reserve University, Cleveland, OH (US); University Hospitals Cleveland Medical Center, Cleveland, OH (US); The United States Government as Represented by The Department of Veteran Affairs, Washington, DC (US)

(72) Inventors: Charlems Alvarez Jimenez, Bogotá (CO); Satish E. Viswanath, Beachwood, OH (US); Joseph E. Willis, Shaker Heights, OH (US); Sofiya Vyshnya, Cleveland, OH (US)

(21) Appl. No.: 19/549,101

(22) Filed: Feb. 25, 2026

Related U.S. Application Data
(60) Provisional application No. 63/777,440, filed on Mar. 25, 2025.

(51) Int. Cl. G06T 7/00; G06T 7/40; G06T 7/60; G16H 50/20

(52) U.S. Cl. CPC G06T 7/0012; G06T 7/40; G06T 7/60; G16H 50/20; G06T 2207/20081; G06T 2207/20084; G06T 2207/30028; G06T 2207/30096

(57) ABSTRACT

Systems, methods, and circuitries are provided for generating a tumor regression grade for a patient. In one example, a method includes extract a cell-related feature for an individual cell represented in image data corresponding to a rectal tumor in a patient; extracting a tissue-related feature for tissue represented in the image data; and determining a tumor regression grade for a patient based on the cell-related feature and the tissue-related feature.

### CROSS-REFERENCE TO RELATED APPLICATIONS

This application claims the benefit of priority from U.S. Provisional Patent Application Ser. No. 63/777,440, filed on Mar. 25, 2025, and entitled COMPUTATION ANALYSIS OF RECTAL TUMOR FEATURES TO DETERMINE RESPONSE TO CHEMORADIATION, the contents of which are hereby incorporated by reference in their entirety for all purposes.

### FEDERAL FUNDING INFORMATION

This invention was made with government support under CA280981 awarded by the National Institutes of Health and 1101BX006439 awarded by the Department of Veterans Affairs. The government has certain rights in the invention.

### BACKGROUND

Rectal cancer is a kind of colorectal cancer that originates in the rectum that can develop from polyps in the rectal lining that become cancerous over time. Chemoradiation therapy may be used to treat rectal cancer, particularly for locally advanced tumors before surgery.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal circuitry of another element may be implemented as an external circuitry and vice versa. Furthermore, elements may not be drawn to scale.

FIG. 1 illustrates some embodiments of an apparatus comprising a machine learning circuitry configured to generate a signature model that generates a tumor regression grade for a patient based on a combination of cell-related features and tissue-related features, in accordance with various aspects described.

FIG. 1A illustrates an example experimental workflow used to generate the signature model of FIG. 1, in accordance with various aspects described.

FIG. 2 illustrates some embodiments of an apparatus configured to generate a tumor regression grade for a patient by evaluating the signature model of FIG. 1, in accordance with various aspects described.

FIG. 3 is a flow diagram outlining an example method for generating a tumor regression score for a patient based on cell-related features and tissue-related features of the patient, in accordance with various aspects described.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Accurate identification of pathologic complete response (pCR) after neoadjuvant chemoradiation (NAC) remains a major challenge in rectal cancer management. For patients with locally advanced rectal cancer, the standard of care involves NAC to reduce tumor burden followed by total mesorectal excision. Histopathology of surgical resection specimens provides the most reliable assessment of treatment response by capturing high resolution tissue and cellular detail. However, visual interpretation is hindered by inter-observer variability even when utilizing standardized scoring systems, limiting bot reproducibility and accuracy. This highlights a need for robust image-derived markers that can reliably identify pCR on resection pathology to guide individualized follow-up in rectal cancers.

Mining quantitative image patterns from digitized pathology slides offers a promising avenue for developing objective descriptors of NAC response. Handcrafted features that capture texture, shape, and intensity on pathology have shown strong association with tumor phenotype, aggressiveness, and patient outcomes in multiple other cancers. A recent advance in digital pathology has been the development of foundation models (FMs) that learn complex, high-level representations of tissue architecture via extensive multi-organ cohorts, and characterization of tumor microenvironment phenotypes in multiple other cancers. Such models could potentially help capture complex tissue architectural patterns linked to NAC response on digitized rectal pathology. However, their complexity makes FMs difficult to interpret, posing challenges for clinical adoption.

Described herein are systems, methods, and circuitry that generate a tumor regression grade (TRG) for a patient based on both cell-related features (e.g., handcrafted features related to texture, shape, or intensity of individual tumor cells) and tissue-related features (e.g., features related to tissue architectural patterns as represented by embeddings generated by a foundation model). The terms cell-related feature and tissue-related feature are used as a shorthand notation for general categories of radiomic features as will be described in more detail below.

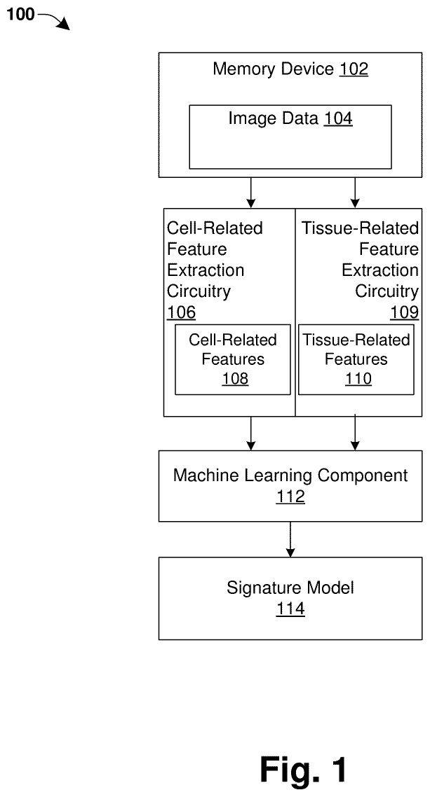

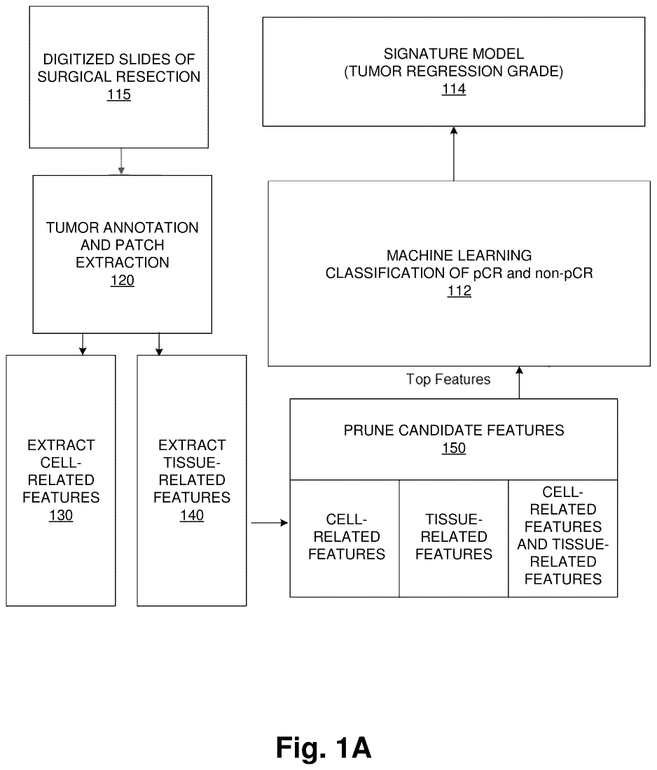

FIG. 1 illustrates some embodiments of an apparatus 100 comprising a machine learning circuitry 112 used to generate, based on image data from a cohort of rectal cancer patients, a signature model 114 that determines a TRG for a new patient based on a plurality cell-related features and tissue-related features extracted from a digitized image data related to the patient's tumor.

The apparatus 100 comprises a memory device 102 configured to store image data 104. The image data 104 comprises digitized images of one or more rectal tissue samples from a cohort of patients that have or are suspected to have rectal cancer. In some embodiments, the image data 104 comprises digitized whole image data from the one or more rectal tissue samples of the patients. Further, the image data 104 may be, for example, a tissue biopsy of a patient that has rectal cancer and is pre-treatment (e.g., pre-CRT treatment) or of a post-treatment resection of a rectal tumor. In various embodiments, the image data 104 may comprise one or more segmented digitized images of tissue taken from a tumor in and/or on the patient's rectum. In some embodiments, the one or more segmented digitized images of the tissue identifies one or more regions of interest (ROI) that may, for example, correspond to one or more tumors on the patient's rectum. The memory device 102 may, for example, comprise electronic memory (e.g., solid state memory, static random-access memory (SRAM), dynamic random-access memory (DRAM), and/or the like).

### Cell-Related Features

The image data 104 may, for example, further comprise image data corresponding to detected and classified individual cells in the one or more regions of interest. The detected and classified cells may be taken from the one or more tumors on the patient's rectum. In yet further embodiments, a cell detection tool (not shown) is coupled between the memory device 102 and a cell-related feature extraction circuitry 106. The cell detection tool is configured to detect and classify the one or more individual cells in the one or more regions of interest. In some embodiments, the cell detection tool is configured to determine a cell location, nuclei segmentation, and/or a cell type of each of the one or more individual cells. The one or more individual cells may be classified as one of several different types of cells, including neoplastic cells, lymphocyte cells, or fibroblast cells.

The cell-related feature extraction circuitry 106 is configured to extract a plurality of cell-related features 108 from the image data 104. The plurality of cell-related features 108 may, for example, include texture features, intensity features, graph-based features, or cellular morphology features for an individual cell. In some embodiments, the cell-related feature extraction circuitry 106 is configured to extract the texture features, intensity features, graph-based features, or cellular morphology features from the one or more regions of interest in the image data 104. In various embodiments, the plurality of texture features and/or the plurality of intensity features are extracted from the one or more individual cells detected and classified by the cell detection tool.

In some embodiments, the feature extraction circuitry 106 is configured to extract the plurality of cell-related features 108 from HSV (Hue, Saturation, Value) color space data and LAB (L: Lightness, A: Red/Green value, B: Blue/Yellow Value) color space data of the image data 104. In various embodiments, the feature extraction circuitry 106 is configured to convert RBG (Red, Blue, Green) image data from the image data 104 into the HSV color space data and the LAB color space data. It has been appreciated that the HSV color space data and the LAB color space data may provide good cell characterization. In various embodiments, the plurality of cell-related features 108 include first order statistics (e.g., mean, standard deviation, skewness and kurtosis) and second order statistics. The second order statistics may, for example, include Haralick descriptors (e.g., 1. Angular Second Moment, 2. Contrast, 3. Correlation, 4. Sum of Squares: Variance, 5. Inverse Difference Moment, 6. Sum Average, 7. Sum Variance, 8. Sum Entropy, 9. Entropy, 10. Difference Variance, 11. Difference Entropy, 12. Information Measure of Correlation 1, 13. Information Measure of Correlation 2, and 14. Maximal Correlation Coefficient). In some embodiments, the plurality of cell-related features 108 include features from one or more neoplastic cells, one or more lymphocyte cells, and one or more fibroblast cells from the image data 104 from the patient. For example, the plurality of cell-related features 108 may include features associated with neoplastic cells (e.g., including Haralick Variance & Information Measure of Correlation), lymphocyte cells (e.g., including Haralick Information Measure of Correlation & Histogram Kurtosis), and/or fibroblast cells (e.g., including Haralick Correlation & Sum Average).

Graph-based cell-related features may include, for example, voronoi graphs, cluster graphs, minimum spanning trees, and so on. Morphology-related features may include, for example, size, shape, elongation, and so on.

### Tissue-Related Features

Tissue-related feature extraction circuitry 109 is configured to extract features related to tissue architecture, referred to as tissue-related features 110. The tissue-related feature extraction circuitry may be, for example, an instantiation of a foundation model that generates embeddings for image data. The respective embeddings provide a classification of a portion of an image along many different dimensions.

A machine learning circuitry 112 is configured to generate a signature model 114 that will generate a TRG for a patient based on at least one cell-related feature 108 and at least one tissue-related feature 110 extracted from the patient's image data. In some embodiments, the machine learning circuitry 112 comprises one or more machine learning models trained to generate the signature model 114 based on the at least one cell-related feature 108 and/or the at least one tissue-related feature 110. The signature model 114 may generate a TRG for the patient. By utilizing the cell-related feature(s) 108 and/or the tissue-related feature(s) 110, the signature model 114 may generate an accurate and repeatable TRG, thereby improving patient care. For example, if the signature model 114 accurately characterizes the patient's TRG as indicating that the patient may have a low likelihood of success from standard chemoradiation therapy, then other treatments and/or other forms of chemoradiation therapy may be pursued.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

FIG. 1A is a flow diagram outlining an example experimental workflow that has been used to generate one example of the signature model 114 of FIG. 1 utilizing the apparatus 100. At 115, whole-slide images (WSIs) of surgical resections were collected from a cohort of rectal cancer patients. Samples were stained with hematoxylin and eosin (H&E) and digitized at 20× and 40× magnification (following which all 40× images were downsampled to 20×). Response to NAC was graded using the American Joint Committee on Cancer tumor regression grading (AJCC TRG) system, with patients segregated into two groups: TRG 0-1 (indicating favorable response/pCR) and TRG 2-3 (indicating poor response/non-pCR). Alternatively, the response to NAC may be graded based on tumor stage (TNM).

At 120, the images were tiled into non-overlapping 256×256 pixel patches, and tumor regions were automatically annotated using the Self-Rule to Multi-Adapt (SRA) algorithm.

At 130 candidate cell-related features were extracted from each tile. Each tile was converted from GRB to HSV color space and 13 Haralick descriptors were extracted from the V channel as described above. The types of cell-related features that are extracted for a tile may depend on the type of an individual cell depicted in the tile. Tile-level features were aggregated using summary statistics (e.g., mean, standard deviation, skewness, and kurtosis across all tiles). In this particular example, the extracted cell-related features included angular second moment, contrast, correlation, sum of squares variance, inverse difference moment, sum average, sum variance, sum entropy, entropy, difference variance, difference entropy, information measure of correlation 1, and information measure of correlation 2.

At 140 candidate tissue-related features were extracted. An FM (e.g., ProvGigaPath) was used to generate tissue embeddings for each tile. The generated embeddings (e.g., 1536 dimensions per tile) were averaged across tumor tiles, yielding 1536 different patient-level candidate tissue-related features. Examples of tissue-related features may include texture or appearance of the tissue based on Haralick analysis.

Candidate cell-related features and candidate tissue-related features were individually evaluated to distinguish between pCR and non-PCR patients. Within each feature set, at 150 redundant candidate features were pruned using Spearman correlation by removing candidate features above a certain threshold, yielding a pruned set of cell-related features and a pruned set of tissue-related features. After pruning 3 cell-related features remained and 24 tissue-related features remained.

A combined set was generated that included concatenated pruned cell-related features and pruned tissue-related samples, yielding a total of 27 features selected from the cell-related features and the tissue-related features. The combined set included 24 tissue-related features (embeddings generated by the FM) and three cell-related features.

Each of the three sets of pruned features generated at 150 (cell-related features, tissue-related features, and combined set of features) were provided to machine learning at 112 to generate a signature model for each set of pruned features that best classified between pCR and non-pCR patients. All models were evaluated via random forest classifiers with 80 trees, across 50 iterations of stratified 5-fold cross-validation at the patient level, with favorable response (pCR) as the positive class. Model performance was compared between feature sets during cross-validation using Wilcoxon signed-rank tests. Performance was accessed across 4 metrics: area under the curve (AUC), sensitivity, specificity, and F1 score.

Table 1 below summarizes the performance of the signature model for each feature set in the five metrics. The results in Table 1 illustrate a best-performing model from 10-fold cross-validation for each set of features. The models were selected according to validation performance and evaluated on the hold-out test set.

TABLE 1

| | Features | AUC | Sensitivity | Specificity | F1 Score |
|---|---|---|---|---|---|
| Cell-Related | 3 | 0.83 | 0.67 | 1.00 | 0.80 |
| Tissue-Related | 24 | 0.92 | 0.67 | 0.75 | 0.67 |
| Combined Set | 27 | 0.94 | 0.83 | 0.75 | 0.77 |

Overall, the combined feature model achieved the strongest performance compared to the cell-related or the tissue-related feature sets. The combined feature model also resulted in the best overall sensitivity. While the cell-related model achieved perfect specificity, this came at the cost of lower sensitivity and AUC. In contrast, the combined feature model demonstrated a more balance performance profile across all metrics, which is a critical consideration for clinical deployment where both false positives and false negatives carry meaningful consequences in assessing NAC response.

The performance of the combined feature model was also reflected in qualitative differences in model attention. The cell-related model was found to exhibit relatively diffuse attention across the whole slide, particular in the non-pCR case; consistent with its comparatively lower discriminative performance. By contrast the tissue-related model demonstrated more spatially focused attention in both pCR and non-pCR cases though it predominantly emphasized muscle and connective tissue regions, which may not directly correspond to residual tumor burden. The combined feature model exhibited attention patterns more closely aligned with biologically relevant tissue structures, which are likely most implicated in treatment response. In non-pCR samples, attention was concentrated within tumor-rich regions, suggesting improved localization of residual disease. In pCR samples, attention was directed toward connective tissue and muscle, consistent with fibrosis, scarring, and inflammatory remodeling that often replace eradicated tumor following NAC.

The above findings suggest that while cell-related features and tissue-related features capture complementary aspects of tissue architecture, their combination resulted in enhanced performance in characterizing response to NAC in rectal cancers.

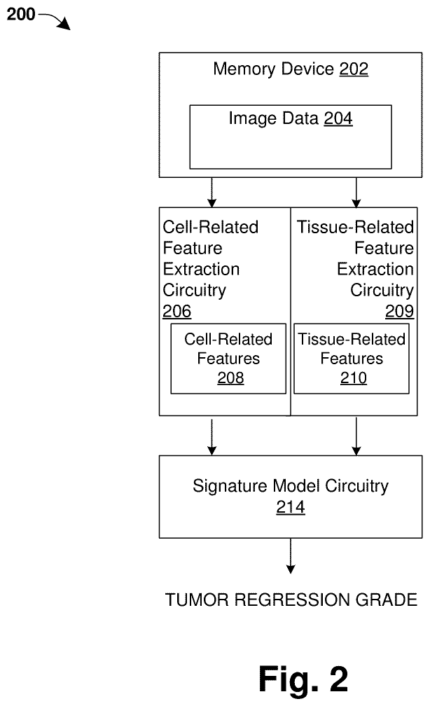

FIG. 2 illustrates some embodiments of an apparatus 200 comprising a signature model circuitry 214 used to generate a TRG for a patient based on image data 204.

The apparatus 200 comprises a memory device 202 configured to store image data 204. The image data 204 comprises digitized images of one or more rectal tissue samples the patient cancer. In some embodiments, the image data 204 comprises digitized whole image data from the one or more rectal tissue samples of the patient. Further, the image data 204 may, for example, be of a patient that has rectal cancer and is pre-treatment (e.g., pre-CRT treatment). In various embodiments, the image data 204 may comprise one or more segmented digitized images of tissue taken from a tumor in and/or on the patient's rectum. In some embodiments, the one or more segmented digitized images of the tissue identifies one or more regions of interest (ROI) that may, for example, correspond to one or more tumors on the patient's rectum. The memory device 202 may, for example, comprise electronic memory (e.g., solid state memory, static random-access memory (SRAM), dynamic random-access memory (DRAM), and/or the like).

### Cell-Related Features

The image data 204 may, for example, include detected and classified individual cells in the one or more regions of interest. The detected and classified cells may be taken from the one or more tumors on the patient's rectum. In yet further embodiments, a cell detection tool (not shown) is coupled between the memory device 202 and a cell-related feature extraction circuitry 206. The cell detection tool is configured to detect and classify the one or more individual cells in the one or more regions of interest. In some embodiments, the cell detection tool is configured to determine a cell location, nuclei segmentation, and/or a cell type of each of the one or more individual cells. The one or more individual cells may be classified, for example, as a neoplastic cell, a lymphocyte cell, or a fibroblast cell.

The cell-related feature extraction circuitry 206 is configured to extract one or more cell-related features 208 from the image data 204. The one or more cell-related features 208 that are extracted may correspond to the cell-related features selected after performance of the experiment outlined with reference to FIG. 1A.

In some embodiments, the feature extraction circuitry 206 is configured to extract the plurality of cell-related features 208 from HSV (Hue, Saturation, Value) color space data and LAB (L: Lightness, A: Red/Green value, B: Blue/Yellow Value) color space data of the image data 204. In various embodiments, the feature extraction circuitry 206 is configured to convert RBG (Red, Blue, Green) image data from the image data 204 into the HSV color space data and the LAB color space data. It has been appreciated that the HSV color space data and the LAB color space data may provide good cell characterization. In various embodiments, the plurality of cell-related features 208 include first order statistics (e.g., mean, standard deviation, skewness and kurtosis) and second order statistics. The second order statistics may, for example, include Haralick descriptors (e.g., 1. Angular Second Moment, 2. Contrast, 3. Correlation, 4. Sum of Squares: Variance, 5. Inverse Difference Moment, 6. Sum Average, 7. Sum Variance, 8. Sum Entropy, 9. Entropy, 10. Difference Variance, 11. Difference Entropy, 12. Information Measure of Correlation 1, 13. Information Measure of Correlation 2, and 14. Maximal Correlation Coefficient). In some embodiments, the cell-related features include one or more of kurtosis Haralick difference entropy, skew Haralick sum entropy, skew Haralick contrast, mean Haralick sum variance, skew Haralick angular second moment, kurtosis Haralick contrast, or skew Haralick information measure of correlation 2.

In some embodiments, the plurality of cell-related features 208 include features from one or more neoplastic cells, one or more lymphocyte cells, and one or more fibroblast cells from the image data 204 from the patient. In some examples, the type of cell-related feature that is extracted for a particular cell depends on the cell's type. For example, cell-related features 208 for a neoplastic cell may include Haralick Variance & Information Measure of Correlation, cell-related features 208 for a lymphocyte cell may include Haralick Information Measure of Correlation & Histogram Kurtosis, and/or cell-related features 208 for a fibroblast cell may include Haralick Correlation & Sum Average.

### Tissue-Related Features

Tissue-related feature extraction circuitry 209 is configured to extract features related to tissue architecture, referred to as tissue-related features 210. The tissue-related feature extraction circuitry may be, for example, an instantiation of a foundation model that generates embeddings for image data. The respective embeddings provide a classification of a portion of an image along many different dimensions. The particular embeddings that are selected for extraction as tissue-related features may include embeddings identified in the experiment outlined with reference to FIG. 1A.

The cell-related feature extraction circuitry 206 provides the extracted cell-related feature(s) to signature model circuitry 214. The tissue-related feature extraction circuitry 209 provides the tissue-related feature(s) 210 to the signature model circuitry 214. The signature model 114 generates a TRG for the patient. By utilizing both cell-related features 108 and tissue-related features 110, the signature model 114 may generate an accurate and repeatable TRG, thereby improving patient care.

The cell-related feature extraction circuitry 206 may include a processor configured to execute stored instructions for extracting the cell-related features from the image data 204. The tissue-related feature extraction circuitry 209 may include a processor configured to execute a foundation model on the image data 204 to generate embeddings that describe tissue architecture. The signature model circuitry 214 may be instantiated by way of a processor executing stored instructions for processing the extracted cell-related features and tissue-related features using a stored signature model. The signature model used by the signature model circuitry 214 may include, for example, a weighted sum of the cell-related features and the tissue-related features, where the weight for each cell-related feature and tissue-related feature is determined by the machine learning circuitry 112 of FIG. 1.

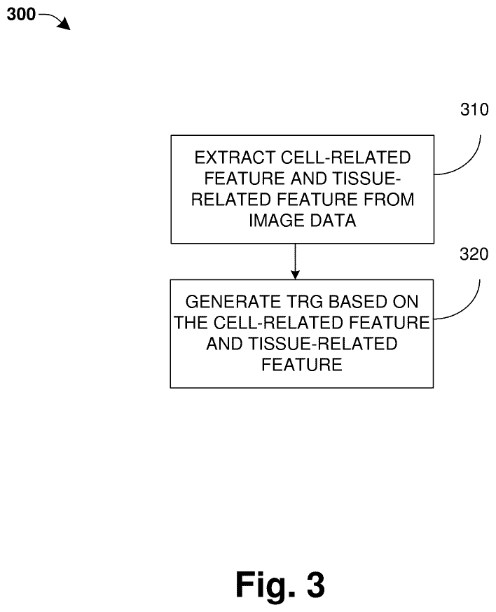

FIG. 3 is a flow diagram outlining an example method 300 for generating a tumor regression grade in a rectal cancer patient. The method may be performed, for example, by the apparatus of FIG. 2 using a signature model 214 generated as described, for example, with reference to FIGS. 1 and 1A. The method includes, at 310, extracting a cell-related feature and a tissue-related feature from image data representing a rectal cancer tumor. In some examples, the cell-related feature and tissue-related feature are selected based on previous analysis of image data of a cohort of rectal cancer patients as described in FIGS. 1 and 1A. Each cell-related feature is extracted from image data corresponding to an individual cell. The tissue-related feature may be extracted using a foundation model tool, such as a ProvGigaPath model.

At 320, the method includes generating a tumor regression grade based on the cell-related features and tissue-related features. In some examples this generating of the TRG for the patient is performed by processing the plurality of cell-related features and tissue-related features using a signature model (e.g., signature model circuitry 214) that generates the TRG for the patient based on the plurality of cell-related features and tissue-related features. The plurality of cell-related features and tissue-related features may include any of the features described with reference to FIG. 1, 1A, or 2.

In some embodiments, the cell-related feature is a texture feature, an intensity feature, a shape feature, or a graph-based feature.

In some embodiments, the method includes classifying the individual cell as a neoplastic cell, a lymphocyte cell, or a fibroblast cell; and extracting the cell-related feature based on the classification of the individual cell. For example, the method may include performing a Haralick analysis of the image data and, when the individual cell is a neoplastic cell, extracting a cell-related feature comprising a variance or an information measure of correlation; when the individual cell is a lymphocyte cell, extracting a cell-related feature comprising an information measure of correlation or a histogram kurtosis; or when the individual cell is a fibroblast cell, extracting a cell-related feature comprising a correlation or a sum average.

In some embodiments, to extract the cell-related feature, the method may include converting the image data into HSV data or LAB data and extract the cell-related feature from the HSV data and/or the LAB data.

In some embodiments the method may include analyzing the image data using a foundation model that characterizes an architectural pattern of a tissue to extract the tissue-related feature. The tissue-related feature may include an embedding produced by the foundation model.

In some embodiments, the method includes inputting the cell-related feature and the tissue-related feature to a signature model that generates the tumor regression grade for the patient based on the cell-related feature and the tissue-related feature.

It can be seen that the described signature for determining a TRG in patients with rectal cancer yields improved medical characterization of the disease in patients. By leveraging a novel multimodal feature interrogation approach, distinct sets of cell-related and tissue-related features are extracted from image data. Integrating these complementary feature sets yielded improved performance in classifying patients as pCR or non-pCR.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of circuitries or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. An apparatus, comprising:
a memory device configured to store image data, wherein the image data comprises digitized images of one or more rectal tissue samples from a patient;
cell-related feature extraction circuitry configured to extract a cell-related feature for an individual cell represented in the image data;
tissue-related feature extraction circuitry configured to extract a tissue-related feature for tissue represented in the image data; and
signature model circuitry configured to determine a tumor regression grade for the patient based on the cell-related feature and the tissue-related feature.

2. The apparatus of claim 1, wherein the cell-related feature comprises a texture feature, an intensity feature, a shape feature, or a graph-based feature.

3. The apparatus of claim 1, wherein the cell-related feature extraction circuitry is configured to
classify the individual cell as a neoplastic cell, a lymphocyte cell, or a fibroblast cell; and
extract the cell-related feature based on the classification of the individual cell.

4. The apparatus of claim 3, wherein the cell-related feature extraction circuitry is configured to perform a Haralick analysis of the image data and, based on the analysis,
when the individual cell is a neoplastic cell, the cell-related feature extraction circuitry is configured to extract a cell-related feature comprising a variance or an information measure of correlation,
when the individual cell is a lymphocyte cell, the cell-related feature extraction circuitry is configured to extract a cell-related feature comprising an information measure of correlation or a histogram kurtosis, or
when the individual cell is a fibroblast cell, the cell-related feature extraction circuitry is configured to extract a cell-related feature comprising a correlation or a sum average.

5. The apparatus of claim 1, wherein the cell-related feature extraction circuitry is configured to
convert the image data into HSV data or LAB data and extract the cell-related feature from the HSV data and/or the LAB data.

6. The apparatus of claim 1, wherein the tissue-related feature extraction circuitry is configured to analyze the image data using a foundation model that characterizes an architectural pattern of a tissue.

7. The apparatus of claim 6, wherein the tissue-related feature comprises an embedding produced by the foundation model that characterizes the tissue.

8. The apparatus of claim 1, wherein the signature model circuitry is configured to input the cell-related feature and the tissue-related feature to a signature model that generates the tumor regression grade for the patient based on the cell-related feature and the tissue-related feature.

9. A method, comprising:
extracting a cell-related feature for an individual cell represented in image data corresponding to a rectal tumor in a patient;
extracting a tissue-related feature for tissue represented in the image data; and
determining a tumor regression grade for a patient based on the cell-related feature and the tissue-related feature.

10. The method of claim 9, wherein the cell-related feature comprises a texture feature, an intensity feature, a shape feature, or a graph-based feature.

11. The method of claim 9, comprising
classifying the individual cell as a neoplastic cell, a lymphocyte cell, or a fibroblast cell; and
extracting the cell-related feature based on the classification of the individual cell.

12. The method of claim 10, comprising
performing a Haralick analysis of the image data and, based on the analysis, and
when the individual cell is a neoplastic cell, extracting a cell-related feature comprising a variance or an information measure of correlation,
when the individual cell is a lymphocyte cell, extracting a cell-related feature comprising an information measure of correlation or a histogram kurtosis, or
when the individual cell is a fibroblast cell, extracting a cell-related feature comprising a correlation or a sum average.

13. The method of claim 9, comprising converting the image data into HSV data or LAB data and extract the cell-related feature from the HSV data and/or the LAB data.

14. The method of claim 9, comprising analyzing the image data using a foundation model that characterizes an architectural pattern of a tissue to extract the tissue-related feature.

15. The method of claim 14, wherein the tissue-related feature comprises an embedding produced by the foundation model.

16. The method of claim 9, inputting the cell-related feature and the tissue-related feature to a signature model that generates the tumor regression grade for the patient based on the cell-related feature and the tissue-related feature.

17. Non-transitory computer-readable medium having instructions stored thereon that, when executed by a processor, cause the processor to perform operations, the operations comprising:
extracting a cell-related feature for an individual cell represented in image data corresponding to a rectal tumor in a patient;
extracting a tissue-related feature for tissue represented in the image data; and
determining a tumor regression grade for a patient based on the cell-related feature and the tissue-related feature.

18. The non-transitory computer-readable medium of claim 17 wherein the operations include
performing a Haralick analysis of the image data and, based on the analysis, and
when the individual cell is a neoplastic cell, extracting a cell-related feature comprising a variance or an information measure of correlation,
when the individual cell is a lymphocyte cell, extracting a cell-related feature comprising an information measure of correlation or a histogram kurtosis, or
when the individual cell is a fibroblast cell, extracting a cell-related feature comprising a correlation or a sum average.

19. The non-transitory computer-readable medium of claim 17, wherein the operations include analyzing the image data using a foundation model that characterizes an architectural pattern of a tissue to extract the tissue-related feature.

20. The non-transitory computer-readable medium of claim 17, wherein the operations include inputting the cell-related feature and the tissue-related feature to a signature model that generates the tumor regression grade for the patient based on the cell-related feature and the tissue-related feature.
